(19) United States
(12) Patent Application Publication
(10) Pub. No.: US 2023/0071397 A1
(43) Pub. Date: Mar. 9, 2023

(54) TREATMENT OF VASOPRESSOR-INDUCED DIGITAL ISCHEMIA WITH BOTULINUM TOXIN A

(71) Applicant: Jason D. Toranto, San Diego (CA)

(72) Inventor: Jason D. Toranto, San Diego (CA)

(21) Appl. No.: 17/468,490

(22) Filed: Sep. 7, 2021

(51) Int. Cl. A61K 38/48; A61K 9/00

(52) U.S. Cl. CPC A61K 38/4893; A61K 9/0019; C12Y 304/24069

(57) ABSTRACT

A method of treating a patient suffering from vasopressor-induced digital ischemia of a hand, finger or thumb comprises providing Botulinum toxin; and injecting the Botulinum toxin in close proximity to one or more blood vessels that supply blood to the hand, finger or thumb, including and not limited to the palmar arch, ulnar and radial arteries. A method of treating a patient suffering from vasopressor-induced digital ischemia of the hand, finger(s), or thumb comprises providing Botulinum toxin; and injecting the Botulinum toxin in close proximity to one or more blood vessels that supply blood to the hand. A method of treating a patient suffering from vasopressor-induced digital ischemia of a foot comprises providing Botulinum toxin; and injecting the Botulinum toxin in close proximity to one or more blood vessels that supply blood to the toe(s) and foot, including and not limited to the plantar arch, anterior tibial/dorsalis pedis and posterior tibial arteries.

### DESCRIPTION OF RELATED ART

The disclosed technology relates generally to the treatment of ischemia, and more particularly some embodiments relate to the treatment of iatrogenic digital ischemia.

### SUMMARY

In general, one aspect disclosed features a method of treating a patient suffering from vasopressor-induced digital ischemia of a finger or thumb, comprising: providing Botulinum toxin; and injecting the Botulinum toxin in close proximity to one or more blood vessels that supply blood to the hand, finger or thumb, including and not limited to the palmar arch, ulnar and radial arteries.

Embodiments of the method may include one or more of the following features. In some embodiments, the Botulinum toxin is selected from the group consisting of: onabotulinum toxin A; abobotulinum toxin A; and incobotulinum toxin A. Some embodiments comprise providing an injection device having a 25 or 27 gauge needle that is 1-1.5 inches in length; and injecting the Botulinum toxin using the injection device. In some embodiments, injecting the Botulinum toxin comprises: introducing the needle from a palmar-dorsal perspective. In some embodiments, injecting the Botulinum toxin comprises: introducing the needle from a distal-proximal perspective. Some embodiments comprise reconstituting 100 units of the Botulinum toxin with 2.0 or 2.5 mL of preservative-free saline.

In general, one aspect disclosed features method of treating a patient suffering from vasopressor-induced digital ischemia of the hand, fingers or thumb, comprising: providing Botulinum toxin; and injecting the Botulinum toxin in close proximity to one or more blood vessels that supply blood to the hand, fingers or thumb.

Embodiments of the method may include one or more of the following features. In some embodiments, the Botulinum toxin is selected from the group consisting of: onabotulinum toxin A; abobotulinum toxin A; and incobotulinum toxin A. Some embodiments comprise providing an injection device having a 25 or 27 gauge needle that is 1-1.5 inches in length; and injecting the Botulinum toxin using the injection device. In some embodiments, injecting the Botulinum toxin comprises: introducing the needle from a palmar-dorsal perspective. In some embodiments, injecting the Botulinum toxin comprises: introducing the needle from a distal-proximal perspective. Some embodiments comprise reconstituting 100 units of the Botulinum toxin with 1.0, 2.0, or 2.5 mL of preservative-free saline. In some embodiments, injecting the Botulinum toxin comprises: injecting the Botulinum toxin in close proximity to at least one of: the radial artery, the ulnar artery, or the palmar arch.

In general, one aspect disclosed features a method of treating a patient suffering from vasopressor-induced digital ischemia of a foot, comprising: providing Botulinum toxin; and injecting the Botulinum toxin in close proximity to one or more blood vessels that supply blood to the toes and foot, including and not limited to the plantar arch, anterior tibial/dorsalis pedis and posterior tibial arteries.

Embodiments of the method may include one or more of the following features. In some embodiments, the Botulinum toxin is selected from the group consisting of: onabotulinum toxin A; abobotulinum toxin A; and incobotulinum toxin A. Some embodiments comprise providing an injection device having a 25 or 27 gauge needle that is 1-1.5 inches in length; and injecting the Botulinum toxin using the injection device. In some embodiments, injecting the Botulinum toxin comprises: introducing the needle from a plantar-dorsal perspective. In some embodiments, injecting the Botulinum toxin comprises: introducing the needle from a distal-proximal perspective. Some embodiments comprise reconstituting 100 units of the Botulinum toxin with 1.0, 2.0, or 2.5 mL of preservative-free saline.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 illustrates the left hand of a patient suffering from Vasopressor-Induced Digital Ischemia (VIDI).

FIG. 2 illustrates the treatment of the fingers of the left hand of a patient with Botulinum Toxin A according to some embodiments of the disclosed technologies.

FIG. 3 illustrates the treatment of the left hand of a patient with Botulinum Toxin A according to some embodiments of the disclosed technologies.

The figures are not exhaustive and do not limit the present disclosure to the precise form disclosed.

### DETAILED DESCRIPTION

Ischemia is a state of inadequate oxygen supply to an organ or part of the body. Since in the human body oxygen is transported via the blood, this typically results from an inadequate blood supply to the organ/body part. Digital ischemia is an inadequate blood supply to one or more fingers and/or toes, and, in the extreme, can result in amputation of one or more digits.

In some cases, digital ischemia is caused by one of several pathologies. For example, digital ischemia may be caused by a vasospastic disorder, which are spasms in the arteries that cause them to narrow and reduce blood flow. One common vasospastic disorder is Raynaud's phenomenon, where the vasospasms are a response to cold or stress. Botulinum Toxin A (Botox) has been used to treat digital ischemia associated with Raynaud's phenomenon.

In other cases, digital ischemia is not caused by a pathology. For example, digital ischemia may be iatrogenic. That is, digital ischemia may be a side effect of a medical treatment. For example, critically ill patients with low blood pressure may be given medication to increase their blood pressure, thereby increasing oxygen delivery to the vital organs. This family of medications are called “vasopressors”. These vasopressors are non-selective in their mechanism of action and cause the constriction of blood vessels throughout the body. This includes the enteric (gut) circulation, the heart, and even the digits of the upper and lower extremities. We describe this ischemia of the digits both in terms of the mechanism and the effect, namely Vasopressor-Induced Digital Ischemia “VIDI”. When VIDI is not recognized and/or the vasopressor dose is not decreased (because the treatment strategies that exists at present do not work), the patient progresses to have symmetrical peripheral gangrene (SPG), which effectively means the patient experiences limb loss. It merits emphasis, again, that this pathology is iatrogenic in nature and therefore this pathology is caused by the doctor.

VIDI stands in stark contrast to the majority of other forms of digital ischemia. With the other forms, there is pathology that is either systemic or specific to the hand that causes the ischemia. In the circumstance of VIDI, there is no “pathology”. Rather, the patient has pathology that necessitates the use of vasopressors (e.g., systemic inflammatory response syndrome (SIRS), shock, etc.), and the digital ischemia is an untoward side-effect of the intervention, namely the use of vasopressors.

There remains no description of efficacious treatment for VIDI. There is one paper that attempts to create a categorization of VIDI and, importantly, names botulinum toxin as a treatment in the most limited cases. The paper goes on to describe a patient treated with botulinum toxin (and a number of other “strategies”) whose VIDI progresses to amputation. The treatment of VIDI with botox in this circumstance failed. In a separate report, the use of botox is mentioned at a dose of 50 units per hand and, again, it failed. Many of the other treatments are “supportive”, which means hand warming, watchful waiting and prayer, and attempting to decrease the use of the vasopressors as much as possible. While there are medicines that have been used/recommended—e.g. nitropaste, sildenafil, antiplatelet agents—none of these have demonstrated true efficacy, which is why the mainstay of treatment remains to decrease the vasopressors. In many circumstances, as above, the unfortunate result is digital amputation. This does not have to be the case. Botulinum toxin has a vigorous capacity to dilate the vascular bed of the hand and toes, thereby improving local oxygen delivery and salvaging digits. The key, however, is that the patient must be treated with an adequate dosage and injection method to counteract the potency of the vasopressors. The inventor has demonstrated this principle in a recent patient. To the best of his knowledge, this is the first and only description of this pathologic entity being efficaciously treated in a manner that specifically counteracts the side-effects of the vasopressors.

As described above, the literature is limited to the presence of digital ischemia in the hand/fingers. The feet and toes are not mentioned in the literature, and there is no description in the literature of efficacious treatment for the feet/toes. The lower extremity is demonstrably different from the upper extremity in countless ways, but perhaps the most important here is that there is a greatly increased distance from the heart to the toes as opposed to the hands. In other words, the further the blood has to travel to deliver oxygen to the tissues, the greater the likelihood of ischemia. This highlights why the absence of a description in the literature of botulinum toxin being used to treat ischemia of the feet/toes is simultaneously shocking and a part of this patent application.

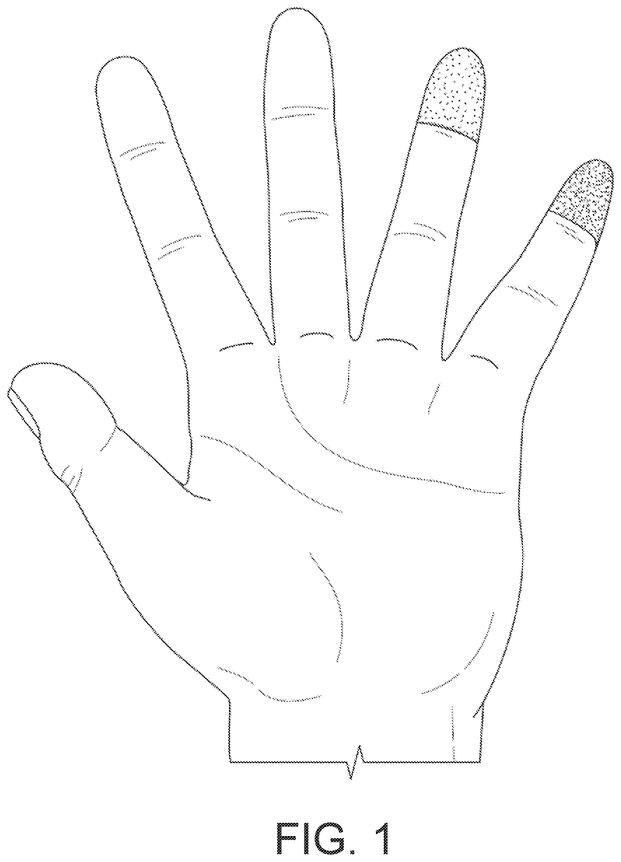

FIG. 1 illustrates the left hand of a patient suffering from VIDI. As can be seen in FIG. 1, the fourth and fifth digits of the patient have become discolored due to inadequate oxygen supply resulting from inadequate blood flow to those digits. Without treatment, those digits may be lost.

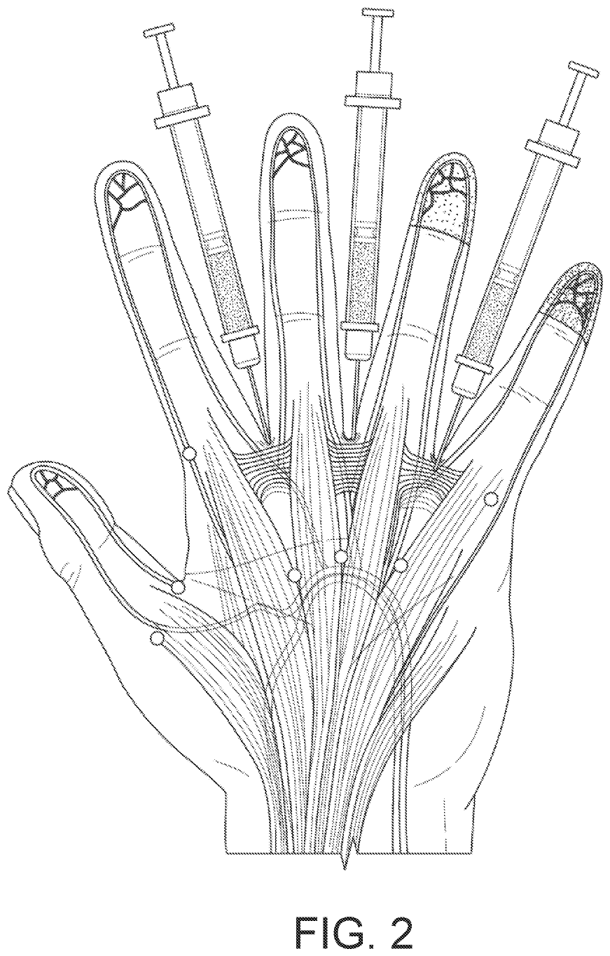

FIG. 2 illustrates the treatment of the fingers of the left hand of a patient with Botulinum Toxin A (Botox) according to some embodiments of the disclosed technologies. The Botox may be introduced in close proximity to the affected blood vessels. The Botox may be introduced at any of the locations indicated in FIG. 2.

Botulinum toxin has a potent vasodilatory effect on the local vascular bed. Botulinum toxin causes vasodilation through at least two mechanisms: (1) by preventing the release of norepinephrine at the neuromuscular junction, and (2) by blocking a phospholipase D pathway for the recruitment of alpha 2c receptors, which are required for vasoconstriction. The improved tissue perfusion achieved with the disclosed treatments is significant, occurs within hours, and may be confirmed by Laser Doppler Flowmetry. However, while the indications for botox include various therapeutic and cosmetic indications, they do not include the treatment of ischemia.

In the hand, the Botox (e.g., onabotulinum toxin A) may be injected in the region around the neurovascular bundles in the region of the metacarpophalangeal joints. Each finger has a dual-vessel blood supply, one on the radial side and one on the ulnar side. However, the ring finger and small finger share a branch vessel off the palmar arch. Therefore, only seven injections are needed to treat all five fingers. The needle may be introduced from a palmar-dorsal perspective, for example at the seven sites indicated by circles in FIG. 2. Alternatively, the needle may be introduced from a distal-proximal perspective. That is, the needle may be introduced in the interspace between two fingers and then moved towards the wrist to reach the introduction sites, as illustrated with the three syringes in FIG. 2. Although three syringes are shown in FIG. 2 for purposes of explanation, it should be understood that only one syringe is needed, though the inventor uses a technique that involves two syringes per hand based on the favored dilution technique. Prior to injection, the plunger is retracted slightly to check for blood in order to avoid intravascular injection.

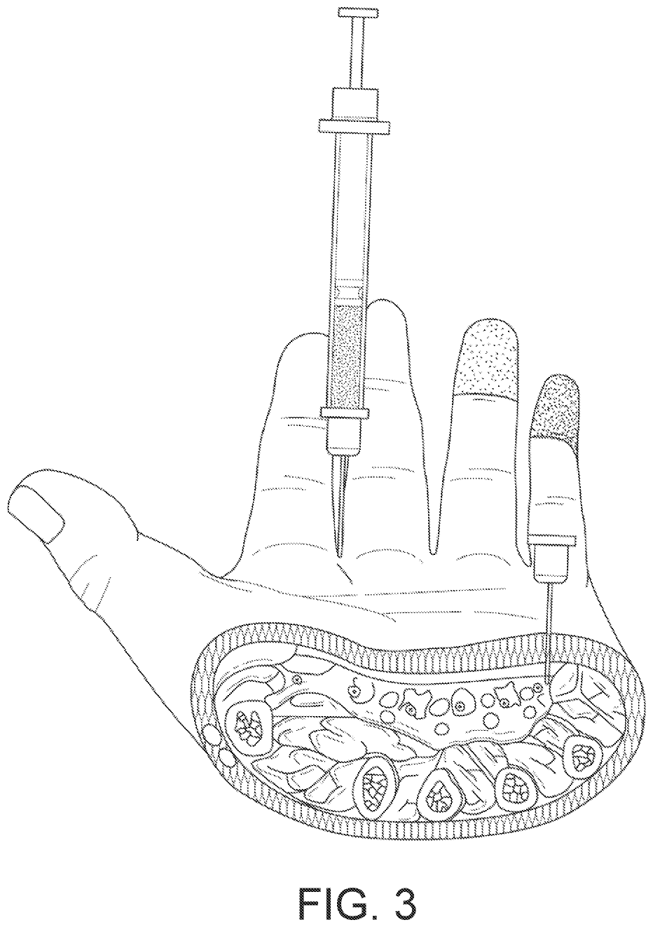

On some occasions it may be desirable to treat the palm of the hand. FIG. 3 illustrates the treatment of the left hand of a patient with Botulinum Toxin A according to some embodiments of the disclosed technologies. The hand is supplied by two vessels: the radial and ulnar arteries. Therefore, injections may be performed near the wrist on either side, as shown in FIG. 3. The Botulinum Toxin A may be introduced in close proximity to the radial artery, the ulnar artery, and/or the palmar arch.

The injection device used to administer the Botulinum Toxin A may have a 25 or 27 gauge needle, which may be 1-1.5 inches in length. The needle may be introduced from the volar aspect of the forearm to address the radial and ulnar arteries.

In some embodiments, 100 units of Botox may be reconstituted with 0.9% sodium chloride with preservative at a dilution of 6-11 cc per 100-unit vial. In some embodiments, 100 units of Botox may be reconstituted with 2.0 or 2.5 mL of preservative-free saline. In some embodiments, other concentrations may be used. For example, in some embodiments, 100 units of Botox may be reconstituted with 1.0 mL of preservative-free saline. Each hand may be treated with 100 units of reconstituted Botox.

Similar techniques may be applied to other distal sites of distal circulation affected by vasopressor-induced ischemia. For example, these techniques may be applied to the foot, and to other sites.

In some embodiments, other forms of botulinum toxin may be employed. These other forms may include, but are not limited to, the following: Dysport (abobotulinum toxin A) and Xeomin (incobotulinum toxin A).

The described treatments possess numerous and significant advantages. The treatments have limited side effects and are safe, even for critically-ill patients under intensive care. Furthermore, the treatments are inexpensive, quick, and minimally invasive. The treatments take effect quickly, perhaps even within fifteen minutes. Finally, the treatments are long-lasting, lasting at least up to 30 days.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. Adjectives such as “conventional,” “traditional,” “normal,” “standard,” “known,” and terms of similar meaning should not be construed as limiting the item described to a given time period or to an item available as of a given time, but instead should be read to encompass conventional, traditional, normal, or standard technologies that may be available or known now or at any time in the future. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A method of treating a patient suffering from vasopressor-induced digital ischemia of a hand, finger, or thumb, comprising:
providing Botulinum toxin; and
injecting the Botulinum toxin in close proximity to one or more blood vessels that supply blood to the hand, finger, or thumb, including and not limited to the palmar arch, ulnar and radial arteries.

2. The method of claim 1, where the Botulinum toxin is selected from the group consisting of:
onabotulinum toxin A;
abobotulinum toxin A; and
incobotulinum toxin A.

3. The method of claim 1, further comprising:
providing an injection device having a 25 or 27 gauge needle that is 1-1.5 inches in length; and
injecting the Botulinum toxin using the injection device.

4. The method of claim 3, wherein injecting the Botulinum toxin comprises:
introducing the needle from a palmar-dorsal perspective.

5. The method of claim 3, wherein injecting the Botulinum toxin comprises:
introducing the needle from a distal-proximal perspective.

6. The method of claim 1, further comprising:
reconstituting 100 units of the Botulinum toxin with 2.0 or 2.5 mL of preservative-free saline.

7. A method of treating a patient suffering from vasopressor-induced digital ischemia of a hand, comprising:
providing Botulinum toxin; and
injecting the Botulinum toxin in close proximity to one or more blood vessels that supply blood to the hand.

8. The method of claim 7, where the Botulinum toxin is selected from the group consisting of:
onabotulinum toxin A;
abobotulinum toxin A; and
incobotulinum toxin A.

9. The method of claim 7, further comprising:
providing an injection device having a 25 or 27 gauge needle that is 1-1.5 inches in length; and
injecting the Botulinum toxin using the injection device.

10. The method of claim 9, wherein injecting the Botulinum toxin comprises:
introducing the needle from a palmar-dorsal perspective.

11. The method of claim 9, wherein injecting the Botulinum toxin comprises:
introducing the needle from a distal-proximal perspective.

12. The method of claim 7, further comprising:
reconstituting 100 units of the Botulinum toxin with 1.0, 2.0, or 2.5 mL of preservative-free saline.

13. The method of claim 7, wherein injecting the Botulinum toxin comprises:
injecting the Botulinum toxin in close proximity to at least one of:
the radial artery,
the ulnar artery, or
the palmar arch.

14. A method of treating a patient suffering from vasopressor-induced digital ischemia of a foot, comprising:
providing Botulinum toxin; and
injecting the Botulinum toxin in close proximity to one or more blood vessels that supply blood to the toes and foot, including and not limited to the plantar arch, anterior tibial/dorsalis pedis and posterior tibial arteries.

15. The method of claim 14, where the Botulinum toxin is selected from the group consisting of:
onabotulinum toxin A;
abobotulinum toxin A; and
incobotulinum toxin A.

16. The method of claim 14, further comprising:
providing an injection device having a 25 or 27 gauge needle that is 1-1.5 inches in length; and
injecting the Botulinum toxin using the injection device.

17. The method of claim 16, wherein injecting the Botulinum toxin comprises:
introducing the needle from a plantar-dorsal perspective.

18. The method of claim 16, wherein injecting the Botulinum toxin comprises:
introducing the needle from a distal-proximal perspective.

19. The method of claim 14, further comprising:
reconstituting 100 units of the Botulinum toxin with 1.0, 2.0, or 2.5 mL of preservative-free saline.
